(19) United States
(12) Patent Application Publication
(10) Pub. No.: US 2014/0010428 A1
(43) Pub. Date: Jan. 9, 2014

(54) METHOD FOR EXTRACTION OF A DATASET FROM A MEDICAL IMAGE DATASET AND ALSO MEDICAL IMAGING DEVICE

(71) Applicant: SIEMENS AKTIENGESELLSCHAFT, Munich (DE)

(72) Inventor: Sebastian SCHMIDT, Weisendorf (DE)

(21) Appl. No.: 13/927,210

(22) Filed: Jun. 26, 2013

(30) Foreign Application Priority Data
Jul. 9, 2012 (DE) 102012211892.6

(51) Int. Cl. A61B 5/00

(52) U.S. Cl. CPC A61B 5/0033
USPC 382/131

(57) ABSTRACT

A method for extraction of a dataset from a medical image dataset is disclosed. A first image dataset, of a body recorded with a first medical modality, is provided which contains first information about a first organ structure. A second image dataset of the body, created with a second medical modality, is provided which contains second information about the first organ structure and about a second organ structure. The second information does not allow a distinction between the first organ structure and the second organ structure. The first image dataset and the second image dataset are processed together with each other such that it is made possible by the processing to separately identify the first organ structure and the second organ structure in the second image dataset.

### PRIORITY STATEMENT

The present application hereby claims priority under 35 U.S.C. §119 to German patent application number DE 102012211892.6 filed Jul. 9, 2012, the entire contents of which are hereby incorporated herein by reference.

### FIELD

At least one embodiment of the invention generally lies in the field of medical technology, especially in the field of oncological diagnostics, and generally relates to a method for extraction of a dataset from a medical image dataset and/or to a medical imaging device.

### BACKGROUND

In oncological diagnostics a staging is regularly carried out to establish a degree of spread of a tumor. Staging is used inter alia to decide on a corresponding course of therapy for a patient. Many types of tumor, such as breast cancer, prostate cancer, bronchial carcinomas or plasmacytomas belong to the so-called cava type. In the event of a metastasis formation, meaning in the event of a migration of the (primary) tumor into remote tissue, the metastases for cava tumors are most likely to form in the skeleton, brain and lungs, but also in the liver and spleen.

For precise staging of a cancer a number of diagnostic tests are thus carried out on the patient. As well as physical examinations and biopsies, imaging methods or modalities, i.e. imaging examination devices to establish the spread of the disease, are especially important. A number of different modalities are often used for such purposes, for example Positron Emission Tomography (PET) to assess the primary tumor, Magnetic Resonance Tomography (MRT) to examine the brain for metastases, Computed Tomography (CT) for the trunk and Single-Photon Emission Computed Tomography (SPECT) or Gamma camera examinations respectively to search for bone metastases in the skeleton. The disadvantage here is that the patient is subjected to a large number of examinations.

Conventionally skeletal scintigraphy/SPECT, PET, MR and CT examinations are each carried out as a separate examination. Scintigraphy and PET practically do not interfere with each other at all or only slightly here, since the scinti tracers employed for scintigraphy do not appear in the PET image and the activity of the PET radio tracers used in PET decays very rapidly.

Basically a complete staging with a combined MR-PET device is able to be carried out in a single examination, since the MR can represent the morphology similarly to the CT and the SPECT is able to be replaced by the PET. However in such cases a number of practical problems emerge.

To investigate the primary tumor the PET radio tracer Fluorodeoxyglucose (18F) (FDG) is usually administered for PET as a radiopharmaceutical, mostly by injection into an arm vein. FDG is absorbed by cells in a similar way to glucose, but however not further metabolized. This results in an accumulation of FDG the cells, which is especially of advantage in the early diagnosis of cancer diseases. Because of the preferred accumulation within cells, FDG does not produce good results to provide evidence of bone metastases. 18F Sodium fluoride (NaF) is typically used for this purpose, with which the bone metabolism is able to be better represented.

The simultaneous administration of FDG and NaF during a PET examination is possible. This produces an improved presentation of bone metastases. However the “clarity” of the presentation is greatly reduced when this approach is employed, since metastases or soft tissue tumors which concentrate FDG are shown in the same image. This makes a pure assessment of the skeleton in respect of metastases which are diagnostically relevant and thereby worthy of treatment more difficult or impossible. The signal contributions of NaF and FDG are not able to be separated in the PET detector, since both compounds emit photons with identical energy. This means that it is not possible with the PET detector to differentiate between FDG and NaF as the radiation source.

Although MRT is basically suitable for investigation of bone metastases it has the disadvantage for example compared to a bone scintigraphy that a large number of image datasets are created during the investigation, which must all subsequently be assessed. This makes the investigation and evaluation disadvantageously very complex and thus especially time- and cost-intensive.

### SUMMARY

At least one embodiment of the invention is directed to a method which makes it possible to record all the important examinations for the staging in a single imaging examination, and on the other hand allows an isolated observation of the skeletal system in a simple overview as a type of projection image, as is the case in skeletal scintigraphy for example.

Advantageous embodiments and developments are the subject matter of the subclaims. At least one embodiment is directed to a medical imaging device. At least one embodiment is directed to a computer program product for automatically executing the method.

In accordance with at least one embodiment of the method, there is provision for extracting a dataset from a medical image dataset of a body, wherein a first image dataset of a body generated with a first medical modality is provided, which contains first information about a first organ structure, especially a bone structure of the body, wherein a second image dataset of the body generated with a second medical modality is provided, which contains second information about the first organ structure and about a second organ structure, especially a tissue structure, of the body, wherein the second information does not enable a distinction to be made between the first organ structure and the second organ structure, and wherein the first image dataset and the second image dataset are processed with one another such that it is made possible thereby to identify the first organ structure and the second organ structure separately in the second image dataset. This is done in at least one embodiment by overlaying the two image datasets such that, by means of the information about the first organ structure contained in the first image dataset, this first organ structure (e.g. bone) is marked in the further image dataset and is made known, so that the organ structures that were previously not able to be distinguished are able to be distinguished in the second image dataset. The particular advantage achieved by this is that for example tissue structures and bone structures are able to be distinguished in a single combined MRT and PET examination with simultaneous administration of two PET radio tracers.

At least one embodiment of the inventive imaging device is directed to a combined MR-PET device with a simultaneous acquisition of a common medical dataset, comprising a display unit for image display of the image dataset and a control unit for signaling extraction of a dataset from the image dataset in accordance with at least one embodiment of the inventive method. For carrying out a combined MR-PET examination two tracers are administered to the patient, of which one specifically accumulates in bone lesions, for example FDG and NaF. In accordance with at least one embodiment of the inventive method the recorded image dataset is created separated into a dataset of a least one of the tracers, based on anatomical assignments, and a projection image of the bone structure as an overview image of the skeletal system. Through this a separate FDG-PET examination and skeletal scintigraphy is replaced by a single examination with the same significance.

At least one embodiment of the inventive computer program product is directed to a file or a data medium containing an executable program which, on installation on a computer, automatically executes at least one embodiment of the inventive method. The data medium is preferably a diskette or CD-Rom on which a corresponding (installation) file for a correspondingly executable computer program is stored. The computer program product is preferably able to be installed on computers which are part of a medical imaging device.

### BRIEF DESCRIPTION OF THE DRAWINGS

Example embodiments of the invention are explained in greater detail below with reference to the drawings, in which:

FIG. 1 shows a schematic diagram of a combined MR-PET device for examining a patient with a monitor for displaying the recorded image dataset and a computer coupled by a signaling link to the monitor for extraction of a dataset from the image dataset,

FIG. 2 shows a flow diagram of the extraction method of the computer in accordance with a first embodiment, and

FIG. 3 shows a flow diagram of the extraction method of the computer in accordance with the second embodiment.

Parts and variables corresponding to one another are always provided with the same reference characters in all figures.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In accordance with at least one embodiment of the method, there is provision for extracting a dataset from a medical image dataset of a body, wherein a first image dataset of a body generated with a first medical modality is provided, which contains first information about a first organ structure, especially a bone structure of the body, wherein a second image dataset of the body generated with a second medical modality is provided, which contains second information about the first organ structure and about a second organ structure, especially a tissue structure, of the body, wherein the second information does not enable a distinction to be made between the first organ structure and the second organ structure, and wherein the first image dataset and the second image dataset are processed with one another such that it is made possible thereby to identify the first organ structure and the second organ structure separately in the second image dataset. This is done in at least one embodiment by overlaying the two image datasets such that, by means of the information about the first organ structure contained in the first image dataset, this first organ structure (e.g. bone) is marked in the further image dataset and is made known, so that the organ structures that were previously not able to be distinguished are able to be distinguished in the second image dataset. The particular advantage achieved by this is that for example tissue structures and bone structures are able to be distinguished in a single combined MRT and PET examination with simultaneous administration of two PET radio tracers.

This makes it possible, in a simple and low-cost way, for example with a combined medical imaging system such as an MR-PET device, to identify or to present the signal contributions of different organ structures separately.

An image dataset is to be understood in general terms as a dataset from which images can be created, this can for example be a raw dataset in the form of measurement data of a modality or also a raw dataset already edited for image presentation.

A dataset is to be understood below especially as 3D image material of a medical imaging device. The 3D image data of the image material includes a number of discrete, as a rule cubiod, volume elements, called voxels. The image material is digitized and stored in a memory, wherein each voxel is on the one hand assigned a location coordinate of the detected image volume, for spatial assignment, and on the other hand is assigned an intensity value, as a measure for the detected signal intensity in the body area of the corresponding voxel. The intensity value is for example able to be executed linearly or logarithmically as a type of grayscale quantization.

The term information especially designates specific measurement data of the respective medical modality, typically raw data or image data edited for an image presentation from the raw data, which allows information to be provided about the structure of the organ under examination, for example an image of the organ structure.

In a suitable development a tissue dataset is created by removing the first information of the first organ structure from the second image dataset or at least specifically marking it and/or a bone dataset is created by all except the first information of the first organ structure being removed from the second image dataset or at least being specifically marked. Through the separation of the image dataset on the basis of anatomical assignments it is ensured that, from a combined image dataset for example, a pure overview image of the skeletal system or of a tissue system is able to be created.

In an advantageous version a projection dataset of the first organ structure is created by the first information of the first organ structure from the first and second image dataset being processed. The projection dataset is especially preferably created from a processing of the first information and the bone dataset. In a preferred embodiment a projection dataset is created from a joint MR-PET image dataset, which on the one hand contains an image of a bone structure from the MRT dataset and on the other hand an image of bone metastases from the PET dataset. The projection dataset merely contains the voxels from the two datasets which are able to be assigned to the skeletal system. An isolated view of the skeletal system for example is thus made possible from the projection data set in a simple overview as a type of projection image, such as is the case for example in skeletal scintigraphy.

In an example embodiment, first and second image datasets are used, which were essentially created at the same time, i.e. during a common imaging examination. The patient is therefore not subjected to unnecessary stress.

In a suitable development the first image dataset is an MRT dataset, which was created by way of an MRT device. In an alternate embodiment it is however also conceivable for the first image dataset to have been created by way of a different radiography device, such as an x-ray device for example.

In a similarly suitable development, the second image dataset is a PET dataset which was created by means a PET. It is also equally conceivable for the second image dataset to have been created by means of a different emission computed tomography, such as a SPECT for example.

In an example embodiment, the second image dataset was created using different tracers, wherein the tracers exhibit a different specific concentration distribution in the first and second organ structure. Preferably two radio nuclides as contrast media or tracers respectively are administered for recording the second image dataset, of which one concentrates specifically in bone lesions. In an advantageous embodiment FDG and NaF are especially used as tracers for this purpose.

Basically, an example embodiment of the inventive method can be extended to other organ systems without any problems if a tracer displays a very specific concentration distribution in an organ or organ system which makes it possible to separate the signal of this tracer on the basis of an anatomical assignment from that of another tracer essentially administered at the same time. For example tracers with a strong concentration in the brain (e.g. F-DOPA) and a simultaneous administration of other tracers with concentrations outside the brain, for example NaF, are conceivable.

In an advantageous embodiment, a three-dimensional mask is created as an image of the first organ structure by means of a segmentation of the first image dataset. Segmentation is especially understood as the creation of content- or anatomically-contiguous regions by way of a combination of image data or voxels respectively. It is especially advantageous here that in many cases the image for segmenting the skeletal system can be simply calculated from the data of an attenuation correction which is integrated in any event conventionally in the imaging system. Since the skeletal system exhibits the strongest attenuation of organs, the bones are already separately identified in the known methods. For example MR sequences with ultrashort echo times (UTE) can be employed, wherein the bones are able to be identified by way of a subsequent image subtraction.

In a suitable development the segmentation is carried out by registration with an existing atlas.

Registration is especially to be understood as a processing of image data, in which two or more image datasets are mapped onto one another in the best possible way, i.e. to determine geometrical transformations, which move the organ systems presented in the images and if necessary also deform them so that they match spatially as well as possible. Under the transformation the model image of the first image dataset is adapted in accordance with a measure of similarity and if necessary in a restricted manner against the reference image of the atlas. In particular individual voxels are mapped onto a generally-applicable atlas to improve the image quality.

Atlas here is to be understood especially as a digital, anatomical atlas of anatomical landmarks or checkpoints. The underlying voxels of atlas and patient, or of the recorded image dataset respectively, are for example adapted to one another by a grayscale-based registration method, and the landmark positions are transferred from the atlas to the recorded image dataset of the patient. The atlas is especially an atlas of the skeletal system with the bones to be identified, wherein the surfaces of the bones are taken as landmarks.

Since the medullary cavity of the bones is often classified in attenuation correction data as having “soft tissue density”, but can still contain bone metastases, the comparison with the atlas is especially simple and advantageous. As an alternative it is however for example just as conceivable to parameterize a segmentation algorithm such that the medullary cavity is also detected. Through the registration with the atlas the image impression is improved to the extent that for example unsharp areas in the image dataset, which can arise for example under some circumstances by the movement of the patient during image recording, are at least partly sharpened.

In a likewise suitable alternate development the first image dataset has voxels with different signal intensity. To create the three-dimensional mask the first image dataset is segmented depending on a signal threshold, wherein merely the voxels of the first image dataset contribute to the three-dimensional mask depending on the signal intensity below the signal threshold.

In an example embodiment there is therefore provision, with an MR-PET image dataset, to evaluate as bones merely the voxels in the MR datasets which only give a very weak signal below a noise threshold, to create a bone mask. Through this, although all voxels will be “incorrectly” classified as air-filled areas, such as for example gas-filled intestinal loops, since these do not generate any PET signal however these errors do not influence the created tissue, bone and projection datasets.

Preferably one or more PET datasets and one or more MR datasets of a patient are essentially recorded at the same time with a combined MR-PET device. The datasets preferably essentially record an image of the entire body or of the greater part of the body of the patient. For recording the PET dataset two tracers, especially FDG and NaF, have been administered to the patient before the examination. The signal contributions of FDG and NaF are identified and presented separately and individually. Use is made of the effect here that NaF selectively accumulates in the bones, while FDG is instead able to be found in the soft tissue. To this end a three-dimensional mask is first created by means of the MR datasets as an image of the skeletal system with a number of voxels, for example by means of registration with an atlas. On the basis of this mask those voxels from the PET dataset which overlay the voxels of the mask are assigned to the skeletal system and segmented into the bone dataset and all other voxels into the (soft) tissue dataset.

The bone dataset thus essentially represents only the PET voxels of which the signal was created by NaF and the tissue dataset essentially represents those PET voxels of which the signal was created by FDG. Subsequently a projection representation of the skeletal system is generated from the common voxels of the mask of the skeletal system and the NaF-PET voxels of the bone dataset, which shows a comparable image impression to that provided by a Gamma camera or skeletal scintigraphy image. Maximum-Intensity-Projection (MIP) is used for example as a method to create this projection presentation. Through this in an advantageous and simple manner essentially all practical problems mentioned at the start are avoided with a combined MR-PET device.

In a further expedient embodiment of the method, the PET determines an activity distribution of the contrast medium during the creation of the tissue dataset, simulates a signal attenuation as a result of body tissue and calculates the signal strength arriving at a radiation detector. This essentially simulates the beam path of the photons of a gamma camera, i.e. the activity distribution is determined from the PET, the attenuation by bodily tissue lying in the beam path and the signal strength at the radiation detector is calculated as a type of ray tracing. In this case artificial parameters can be accepted, for example a greater attenuation by bones, through which the image impression is improved to the extent that it is easier to distinguish whether a lesion lies dorsally or ventrally on the body.

At least one embodiment of the inventive imaging device is directed to a combined MR-PET device with a simultaneous acquisition of a common medical dataset, comprising a display unit for image display of the image dataset and a control unit for signaling extraction of a dataset from the image dataset in accordance with at least one embodiment of the inventive method. For carrying out a combined MR-PET examination two tracers are administered to the patient, of which one specifically accumulates in bone lesions, for example FDG and NaF. In accordance with at least one embodiment of the inventive method the recorded image dataset is created separated into a dataset of a least one of the tracers, based on anatomical assignments, and a projection image of the bone structure as an overview image of the skeletal system. Through this a separate FDG-PET examination and skeletal scintigraphy is replaced by a single examination with the same significance.

At least one embodiment of the inventive computer program product is directed to a file or a data medium containing an executable program which, on installation on a computer, automatically executes at least one embodiment of the inventive method. The data medium is preferably a diskette or CD-Rom on which a corresponding (installation) file for a correspondingly executable computer program is stored. The computer program product is preferably able to be installed on computers which are part of a medical imaging device.

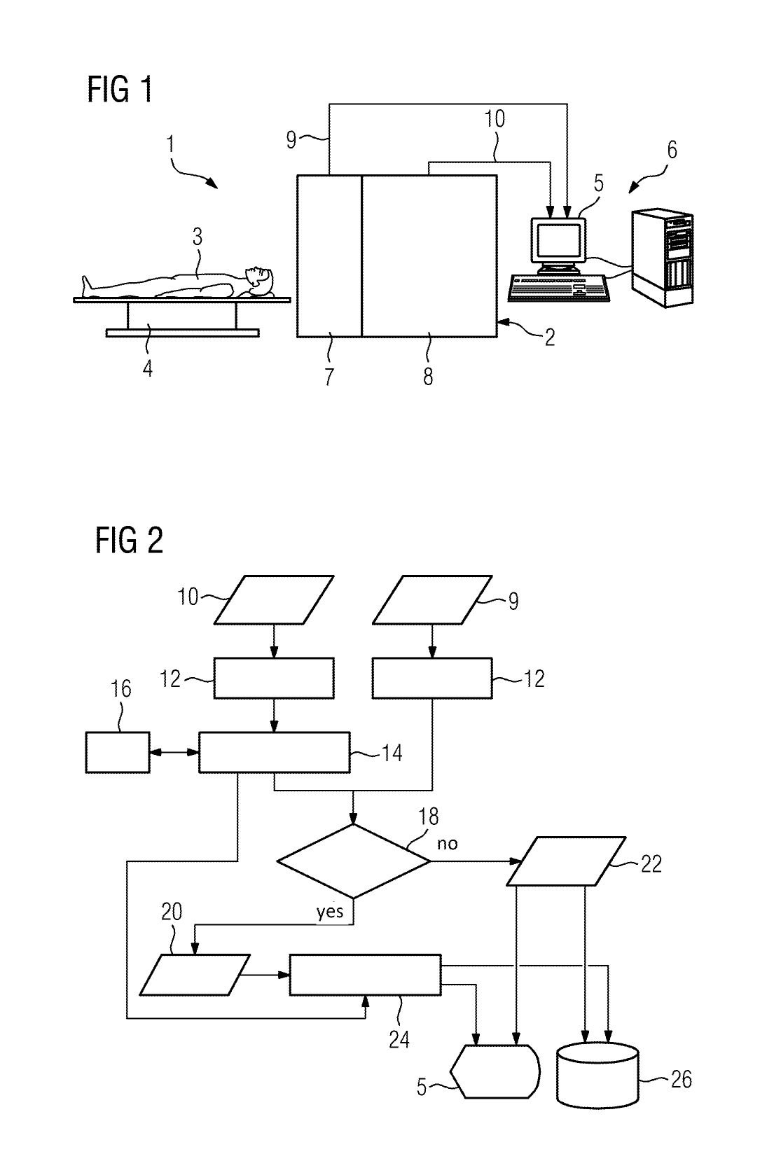

FIG. 1 shows a greatly simplified diagram of the structure and the functioning of an imaging device 1 with a combined MR-PET device 2 for staging a patient 3 to be examined. The patient 3 lies during an examination on a moveable OP table 4. The MR-PET device 2 is coupled by a signaling link to a monitor 5 and a computer 6. The monitor 5 is used for image display of the examination data recorded by the MR-PET device. For examination the patient 3 is essentially able to be moved entirely by means of the OP table 4 into the MR-PET device 2 so that essentially the entire body or the greater part of the body of the patient 3 is able to be examined.

The MR-PET device 2 essentially comprises a PET 7 and an MRT 8, which during a scan each create an image dataset 9, 10. The image datasets 9, 10 are recorded simultaneously during an examination. To carry out a combined MR-PET examination two radionuclides are administered to the patient 3 by means of injection as PET tracers, of which one specifically accumulates in bone lesions, especially FDG and NaF.

During an examination the MR-PET device 2 essentially simultaneously records one or more fluoroscopy datasets, namely the PET dataset 9 of the PET 7 and the MRT dataset 10 of the MRT 8. Both the PET dataset and also the MRT dataset essentially image the entire body or the greater part of the body respectively of the patient 3. The fluoroscopy datasets are sent to the computer 6 and evaluated by this by way of the stored evaluation program, in particular the computer 6 is thus configured by software to extract the signal contributions of FDG and NaF of the PET dataset 9 by way of the MRT dataset 10, identify them separately and present them individually on the monitor 5.

The extraction of the signal contributions by the evaluation program of the computer 6 is explained in greater detail below with reference to FIG. 2.

For extraction of the signal contributions use is made of the effect that NaF accumulates selectively in the bones while FDG tends to be found in soft tissue. To this end, in step 12 “registration/image computation”, the MRT dataset 10 is first registered and a three-dimensional MRT image of the patient 3 with a number of voxels is created from the registered data. The voxels of the MRT image are segmented in the next step 14 by being compared with an atlas 16 of the skeletal system and with the voxels which are able to be assigned to a bone, a pure image of the bone structure is created as a three-dimensional mask. The PET dataset 9 is registered in this version in the known way and a three-dimensional PET image of the patient 3 with a number of voxels is created.

On the basis of the image of the bone structure a check is subsequently performed in step 18 as to whether voxels assigned to a specific same position are contained in both images, i.e. overlay one another. From the PET image those voxels which essentially overlay a voxel of the image in their spatial coordinates are assigned to the skeletal system and segmented jointly into a bone tumor dataset 20, and all other voxels into a soft tissue dataset 22.

The bone tumor dataset 20 thus essentially contains only those PET voxels, of which the signal has been generated by NaF and the soft tissue dataset 22 essentially contains only those voxels, of which the signal has been generated by FDG.

Subsequently, in step 24 a projection presentation of the skeletal system is generated from the bone tumor dataset 20 and the mask, which shows a comparable image impression to that of a Gamma camera or skeletal scintigraphy image. The completed evaluated data is on the one hand stored in a database 26 of the computer 6 and on the other hand is able to be displayed on the monitor 5 for staging.

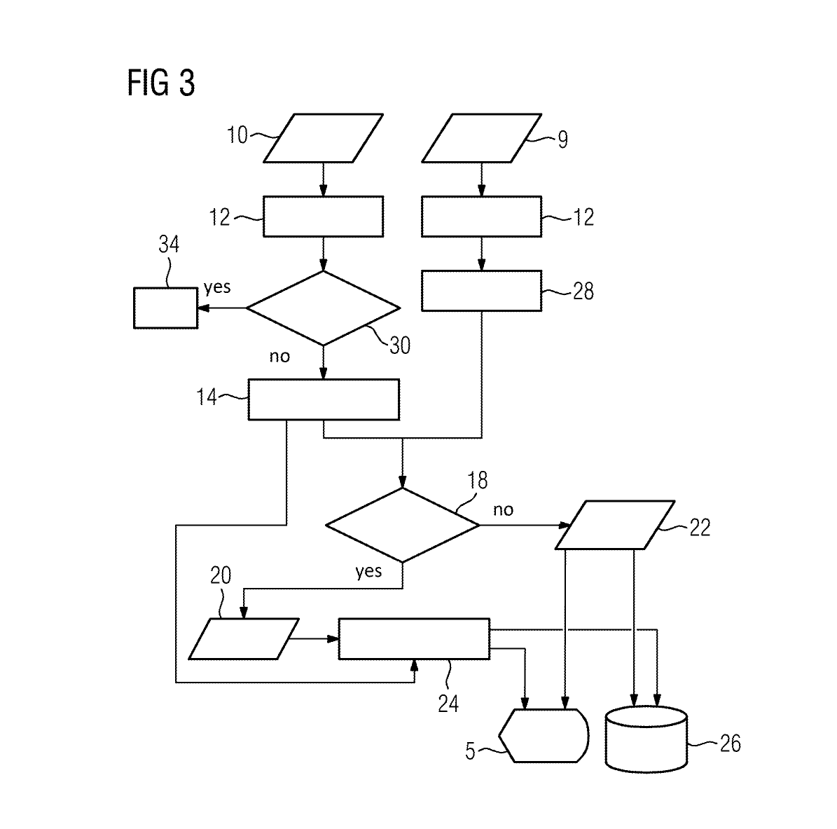

An alternate method for extraction of the signal contributions by the evaluation program of the computer 6 is explained in greater detail below with reference to FIG. 3. The alternate method differs from that described previously essentially through the evaluation of the PET dataset and the MRT dataset.

In this method the PET dataset 9 is initially registered in step 12 and a three-dimensional PET image of the patient 3 with a number of voxels is created. Subsequently, in step 28 the PET image is evaluated in the manner of a ray tracing. To this end initially the activity distribution is determined by evaluation of the location coordinates and signal intensity of each voxel of the PET image. Subsequently a signal attenuation is simulated by the evaluation program by body tissue of the patient 3 lying in the beam path and from this a “corrected” signal intensity is assigned to the voxels. This improves the image impression such that it is easier to distinguish whether a possible lesion lies dorsally or ventrally on the body.

The MRT dataset 10 is registered in step 12 and from the registered data a three-dimensional MRT image of the patient 3 with a number of voxels is created. The assigned signal intensity of each voxel of the MRT image is compared in a next step 30 with a signal threshold, wherein in step 14 only those voxels for creating the image of the bone structure are used of which the signal intensity lies below the signal threshold. Although all voxels of air-filled areas such as for example gas-filled intestinal loops, are “incorrectly” classified by this, since these however do not generate any PET signal and thus do not occur in the PET dataset 9, these errors do not influence the extracted dataset. The remaining voxels are discarded in step 34. The subsequent execution sequence is identical to the sequence described for FIG. 1.

The invention is not restricted to the present exemplary embodiments described. Instead other variants of the invention can also be derived herefrom by the person skilled in the art, without departing from the subject matter of the invention. In particular all the individual features described in connection with the different example embodiments are also able to be combined with one another in a different way without departing from the subject matter of the invention.

1. A method for extraction of a dataset from a medical image dataset, comprising:
providing a first image dataset of a body created with a first medical modality, the first image dataset including first information about a first organ structure of the body;
providing a second image dataset of the body created with a second medical modality, the second image dataset including second information about the first organ structure and about a second organ structure of the body, wherein the second information does not allow a distinction between the first organ structure and the second organ structure;
processing the first image dataset and the second image dataset with one another, the processing making the first organ structure and the second organ structure separately identifiable in the second image dataset.

2. The method of claim 1, further comprising at least one of
creating a tissue dataset by the first information of the first organ structure being removed from, or at least specially marked in the second image dataset, and
creating a bone dataset by all but the first information of the first organ structure being removed from, or at least specially marked in the second image dataset.

3. The method of claim 1, further comprising:
creating a projection dataset of the first organ structure by the first information of the first organ structure from the first and second image dataset being processed.

4. The method of claim 1, wherein first and second image datasets, which are essentially created simultaneously, are used.

5. The method of claim 1, wherein the first image dataset is a magnetic resonance tomography dataset created by a magnetic resonance tomography device.

6. The method of claim 1, wherein the second image dataset is a positron emission tomography dataset created by a positron emission tomography device.

7. The method of claim 6, wherein the second image dataset was created using different tracers, whereby the tracers exhibit a different specific accumulation distribution in the first and second organ structure.

8. The method of claim 1, wherein a three-dimensional mask of the first organ structure is created by ways of a segmentation of the first image dataset.

9. The method of claim 8, wherein the segmentation is performed by registration with an atlas.

10. The method of claim 8, wherein the first image dataset includes voxels with different signal intensity and, to create the three-dimensional mask, the first image dataset is segmented as a function of a signal threshold, wherein only the voxels of the first image dataset contribute to the three-dimensional mask as a function of a signal intensity below the signal threshold.

11. The method of claim 2, wherein, in the creation of the tissue dataset of the positron emission tomograph, an activity distribution of the contrast medium is determined, a signal attenuation based on body tissue is simulated and the signal strength at a radiation detector is calculated.

12. A medical imaging device, including a first and second medical modality with essentially simultaneous acquisition of a joint medical image dataset, comprising:
a display unit configured to display the joint medical image dataset; and
a control unit configured to extract a dataset from the joint medical image dataset via a signal link, the control unit being configured to process a first image dataset of a body, including first information about a first organ structure of the body and created with the first medical modality, and a second image dataset of the body with one another, the second image dataset including second information about the first organ structure and about a second organ structure of the body created with the second medical modality, the processing making the first organ structure and the second organ structure separately identifiable in the second image dataset, wherein the second information does not allow a distinction between the first organ structure and the second organ structure.

13. A computer readable medium containing an executable program which, when installed on a computer, automatically executes the method of claim 1.

14. The method of claim 1, wherein the first organ structure is a bone structure of the body and the second organ structure is a tissue structure.

15. The method of claim 2, further comprising:
creating a projection dataset of the first organ structure by the first information of the first organ structure from the first and second image dataset being processed.

16. The method of claim 2, wherein first and second image datasets, which are essentially created simultaneously, are used.

17. The method of claim 5, wherein the second image dataset is a positron emission tomography dataset created by a positron emission tomography device.

18. The medical imaging device of claim 12, wherein the medical imaging device is a combined MR-PET device.
